(19) United States
(12) Patent Application Publication
(10) Pub. No.: US 2015/0025295 A1
(43) Pub. Date: Jan. 22, 2015

(54) MEDICAL TREATMENT APPARATUS, CONTROL DEVICE AND CONTROL METHOD

(71) Applicant: KABUSHIKI KAISHA TOSHIBA, Tokyo (JP)

(72) Inventors: Kyoka Sugiura, Kawasaki-shi (JP); Yasunori Taguchi, Kawasaki-shi (JP); Takeshi Mita, Yokohama-shi (JP)

(21) Appl. No.: 14/334,897

(22) Filed: Jul. 18, 2014

(30) Foreign Application Priority Data
Jul. 19, 2013 (JP) 2013-150422

(51) Int. Cl. A61N 5/10

(52) U.S. Cl. CPC A61N 5/1049; A61N 5/103
USPC 600/1

(57) ABSTRACT

According to an embodiment, a medical treatment apparatus includes: a first acquirer to acquire a group including five or more pairs of corresponding points on a first perspective image of a subject captured at a first timing and a second perspective image of the subject captured at a second timing; a second acquirer to acquire a first parameter including position/orientation information of an imaging device capturing the first perspective image and conversion information related to a coordinate system of the first perspective image, and acquire a second parameter including position/orientation information of an imaging device capturing the second perspective image and conversion information related to a coordinate system of the second perspective image; a calculator to calculate difference in position of the subject between the first timing and the second timing using the group and the parameters; and a controller to control a subject position using the difference.

### CROSS-REFERENCE TO RELATED APPLICATIONS

This application is based upon and claims the benefit of priority from Japanese Patent Application No. 2013-150422, filed on Jul. 19, 2013; the entire contents of which are incorporated herein by reference.

### FIELD

Embodiments described herein relate generally to a medical treatment apparatus, a control device and a control method.

### BACKGROUND

In radiotherapy, fluoroscopic images of a subject are captured during the treatment planning and during the treatment, and the position of the subject is controlled using these images so that the position of the subject at a time of the actual treatment is consistent with the position as the time of the treatment planning.

For example, images of the subject are captured from two different directions during the treatment planning and during the treatment. Three pairs of corresponding points are then designated on the captured images, the number of which is four in total, and the difference in position of the subject between the time of the treatment planning and the time of the treatment is calculated.

However, because such a conventional technology requires designations of three pairs of corresponding points on the images captured from different directions, the position of one corresponding point in each pair often include some error, so the resultant subject position control is performed less accurately.

### BRIEF DESCRIPTION OF THE DRAWINGS

FIG. 1 is a schematic illustrating a medical treatment apparatus according to a first embodiment;

FIG. 2 is a flowchart illustrating a process performed by the medical treatment apparatus according to the first embodiment;

FIG. 3 is a schematic of an arrangement of a first perspective image and a second perspective image according to the first embodiment;

FIG. 4 is a flowchart illustrating a corresponding point group acquiring process according to the first embodiment;

FIG. 5 is a schematic illustrating a first group according to the first embodiment;

FIG. 6 is a flowchart illustrating a displacement calculating process according to the first embodiment;

FIG. 7 is a schematic for explaining the displacement calculating process according to the first embodiment;

FIG. 8 is a schematic illustrating a medical treatment apparatus according to a second embodiment;

FIG. 9 is a flowchart illustrating a process performed by the medical treatment apparatus according to the second embodiment;

FIG. 10 is a schematic of an arrangement of first to fourth perspective images according to the second embodiment;

FIG. 11 is a flowchart illustrating a corresponding point group acquiring process according to the second embodiment;

FIG. 12 is a schematic illustrating a first group and a second group according to the second embodiment;

FIG. 13 is a flowchart illustrating a displacement calculating process according to the second embodiment; and

FIG. 14 is a block diagram illustrating a hardware configuration of the medical treatment apparatus.

### DETAILED DESCRIPTION

According to an embodiment, a medical treatment apparatus includes a first acquirer, a second acquirer, a calculator, and a controller. The first acquirer acquires a first acquirer that acquires a first group including five or more pairs of corresponding points respectively on a first perspective image of a subject viewed in a first direction at a first timing and a second perspective image of the subject viewed in a second direction at a second timing being different from the first timing. The second acquirer acquires. a second acquirer that acquires a first parameter and a second parameter, the first parameter including position and orientation information of an imaging device that captures the first perspective image and including conversion information related to a coordinate system of the first perspective image, and the second parameter including position and orientation information of an imaging device that captures the second perspective image and including conversion information related to a coordinate system of the second perspective image. The calculator calculates difference in position of the subject between the first timing and second timing using the first group, the first parameter, and the second parameter. The controller controls a position of the subject using the difference.

Various embodiments will be explained in detail with reference to the appended drawings.

### First Embodiment

Explained in a first embodiment is an example in which the amount of rotational movement of a subject from a first point in time to a second point in time is calculated, the position of the subject is controlled using the calculated amount of rotational movement, and the radiotherapy is conducted on the subject of which the position has been controlled. Examples of the radiotherapy include those using particle beam treatment apparatuses that conduct medical treatment with heavy particle beams or proton beams. In the first embodiment, the first point in time is considered to be a point in time at which images of the subject are captured during the planning of radiotherapy, and the second point in time to be a point in time at which images of the subject are captured when the radiotherapy is conducted, but are not limited thereto.

According to the first embodiment, when a displacement of a position of the subject from the first point in time to the second point in time results from rotational movement, the amount of the displacement can be corrected before the actual radiotherapy.

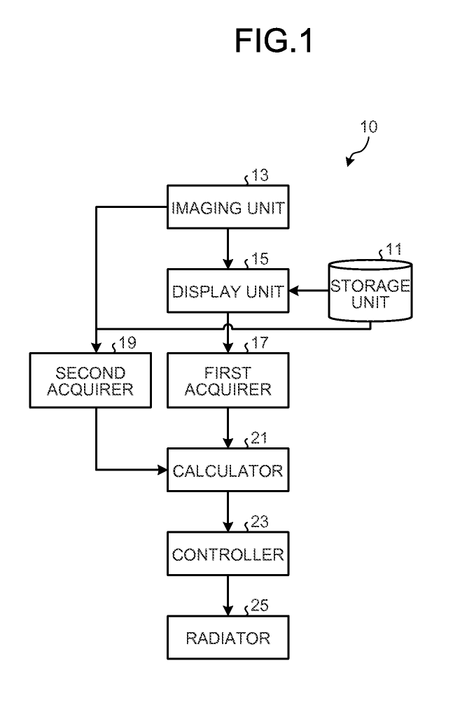

FIG. 1 is a schematic illustrating an example of a configuration of a medical treatment apparatus 10 according to the first embodiment. As illustrated in FIG. 1, the medical treatment apparatus 10 includes a storage unit 11, an imaging unit 13, a display unit 15, a first acquiring unit 17, a second acquiring unit 19, a calculating unit 21, a control unit 23, and a radiation unit 25.

The storage unit 11 stores therein a first perspective image that is a fluoroscopic image of a subject captured at the first point in time from a first direction, position and orientation information related to the position and orientation of an imaging device capturing the first perspective image at the first point in time, and a first parameter including conversion information related to conversions of a normalized coordinate system into a first perspective image coordinate system. The storage unit 11 may be provided as a storage device capable of magnetic, optical, or electrical storage, such as a hard disk drive (HDD), a solid state drive (SSD), a memory card, an optical disc, and a random access memory (RAM).

In the first embodiment, the imaging device that captures the first perspective image is considered to be an imaging unit (not illustrated) that is not the imaging unit 13, which is to be explained later, but may be the imaging unit 13. The imaging device that captures the first perspective image includes a radioactive beam radiation unit that irradiates a radioactive beam, and a sensor that detects the radioactive beam irradiated from the radioactive beam radiation unit and generates a fluoroscopic image of an object (in the first embodiment, the subject) captured with the radioactive beam, for example. Examples of the sensor include a flat panel detector (FPD) and an image intensifier (II). The first parameter can be implemented as a camera parameter (an external parameter or an internal parameter) of the imaging device (more specifically, the radioactive beam radiation unit) that captures the first perspective image at the first point in time, as an example, and can be acquired through calibration of the imaging device that captures the first perspective image.

The imaging device that captures the first perspective image may be provided as a computed tomography (CT) device. In such a case, a digitally reconstructed radiography (DRR) image generated from the volume data captured by the CT device serves as the first perspective image, as an example, and a camera parameter of a virtual camera at the time when the first perspective image is generated serves as the first parameter, as an example.

The imaging unit 13 captures a second perspective image that is a fluoroscopic image of the subject captured at the second point in time that is not the first point in time from a direction approximately the same as the first direction. In the first embodiment, the second point in time is considered to be a point in time subsequent to the first point in time, as mentioned earlier, but without limitation. For the direction approximately the same as the first direction, the same direction as the first direction is assumed, but some error is tolerable. In other words, one of the directions centered about the first direction within a certain error can be used as the direction approximately the same as the first direction. The certain error may be predetermined, or may be visually determined by an operator (radiographer) of the medical treatment apparatus 10 such as a physician.

The imaging unit 13 includes, for example, a radioactive beam radiation unit that irradiates a radioactive beam, and a sensor that detects the radioactive beam irradiated from the radioactive beam radiation unit and generates a fluoroscopic image of an object (in the first embodiment, the subject) from the radioactive beam. Examples of the sensor include an EPD and II.

The display unit 15 displays the first perspective image stored in the storage unit 11 and the second perspective image captured by the imaging unit 13. The display unit 15 may be provided as a display device such as a touch panel display and a liquid crystal display.

The first acquiring unit 17 acquires a first group including five or more pairs of corresponding points on the first perspective image and the second perspective image. The first group represents five or more pairs of corresponding points designated on the first perspective image and the second perspective image displayed on the display unit 15. In other words, the first acquiring unit 17 acquires five or more pairs of corresponding points designated on the first perspective image and the second perspective image displayed on the display unit 15 as a first group.

In the first embodiment, the five or more pairs of corresponding points are explained to be designated on the first perspective image and the second perspective image by an operator of the medical treatment apparatus 10. When the display unit 15 is a touch panel display, the operator of the medical treatment apparatus 10 directly designates and enters the corresponding points on the first perspective image and the second perspective image displayed on the display unit 15. When the display unit 15 is a liquid crystal display, the operator of the medical treatment apparatus 10 designates and enters the corresponding points on the first perspective image and the second perspective image displayed on the display unit 15 using an input device (not illustrated) such as a mouse.

The way in which the five or more pairs of corresponding points are designated on the first perspective image and the second perspective image is not limited to the examples described above. For example, the corresponding points may be designated by allowing the operator of the medical treatment apparatus 10 to designate one point on the first perspective image or the second perspective image, and causing the first acquiring unit 17 to search a point corresponding to the designated point from the other image. The first acquiring unit 17 may use template matching, for example, to search the corresponding point from the other image.

Another possible way is to cause the first acquiring unit 17 to search and designate a characterizing point on one of the first perspective image or the second perspective image, and to search a point corresponding to the designated (searched) characterizing point from the other image and to designate the point as the corresponding point. An Example of the characterizing point includes an edge. The first acquiring unit 17 can search a corresponding point on the other image through template matching, for example, in the same manner as in the earlier example.

The operator of the medical treatment apparatus 10 may also be allowed to correct the position of the characterizing point and the corresponding point searched by the first acquiring unit 17 on the display unit 15.

The second acquiring unit 19 acquires the first parameter, the position and orientation information related to the position and orientation of the imaging unit 13 capturing the second perspective image at the second point in time, and a second parameter including conversion information related to conversions of the normalized coordinate system into a second perspective image coordinate system. The second parameter can be implemented as a camera parameter (external parameter and internal parameter) of the imaging unit 13 (more specifically, that of the radioactive beam radiation unit) at the second point in time, for example, and can be acquired through calibration of the imaging unit 13 capturing the second perspective image. Specifically, the second acquiring unit 19 acquires the first parameter from the storage unit 11, and acquires the second parameter from the imaging unit 13.

The calculating unit 21 calculates difference in position of the subject between the first point in time and the second point in time, using the first group acquired by the first acquiring unit 17, and the first parameter and the second parameter acquired by the second acquiring unit 19. In the first embodiment, the difference is explained to be the amount of rotational movement of the subject from the first point in time to the second point in time (more specifically, the amount of rotation of the subject at the second point in time with respect to the position of the subject at the first point in time).

The control unit 23 controls the position of the subject using the difference calculated by the calculating unit 21. Specifically, the control unit 23 controls to adjust the position of the subject to the position of the subject at the first point in time, using the amount of rotational movement calculated by the calculating unit 21. The control unit 23 controls to rotate the bed on which the subject lies (not illustrated) based on the amount of rotational movement calculated by the calculating unit 21 so that the subject is moved to the position of the subject at the first point in time.

The radiation unit 25 irradiates a therapeutic beam to an affected area of the subject of which the position has been controlled. In the first embodiment, the therapeutic beam is considered to be a radioactive beam, but may also be a heavy particle beam, a proton beam, an X-ray, a gamma ray, and the like. Specifically, the radiation unit 25 irradiates a therapeutic beam in accordance with the irradiation information specified during the treatment planning by the operator of the medical treatment apparatus 10. Examples of the irradiation information include the intensity of the radioactive beam, an area irradiated with the radioactive beam, and an angle at which the radioactive beam is irradiated when the affected area of the subject is irradiated with the therapeutic beam.

In the first embodiment, although the area irradiated with the therapeutic beam, the angle at which the therapeutic beam is irradiated, and the like are determined during the treatment planning, as explained earlier, the radiation unit 25 can output a therapeutic beam to the affected area of the subject more accurately because the control unit 23 controls to adjust the position of the subject to the position of the subject at the first point in time. The coordinate system of the medical treatment apparatus 10 has its origin at the isocenter, for example. The isocenter is at a point of intersection of a plurality of fluoroscopic radioactive beams that are irradiated from different directions to the affected area of the subject.

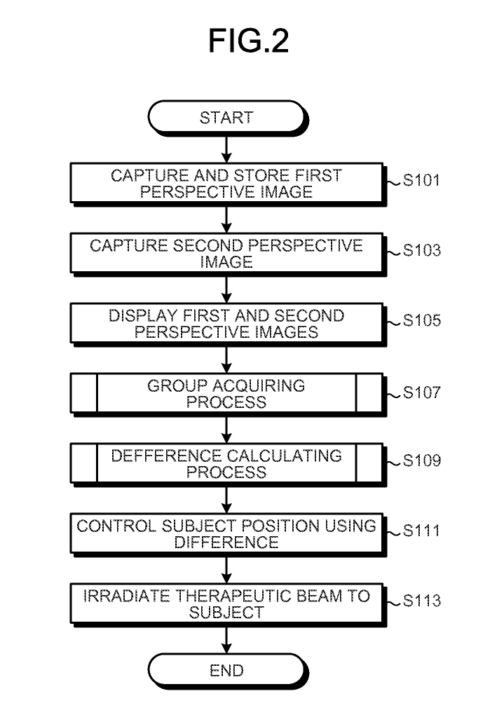

FIG. 2 is a flowchart of an example of a process performed by the medical treatment apparatus 10 according to the first embodiment.

To begin with, the imaging device for capturing the first perspective image captures the first perspective image that is a fluoroscopic image of the subject at the first point in time from the first direction, and the captured first perspective image and the first parameter of the imaging device capturing the first perspective image are stored in the storage unit 11 (Step S101).

The imaging unit 13 then captures the second perspective image that is a fluoroscopic image of the subject at the second point in time from a direction approximately the same as the first direction (Step S103).

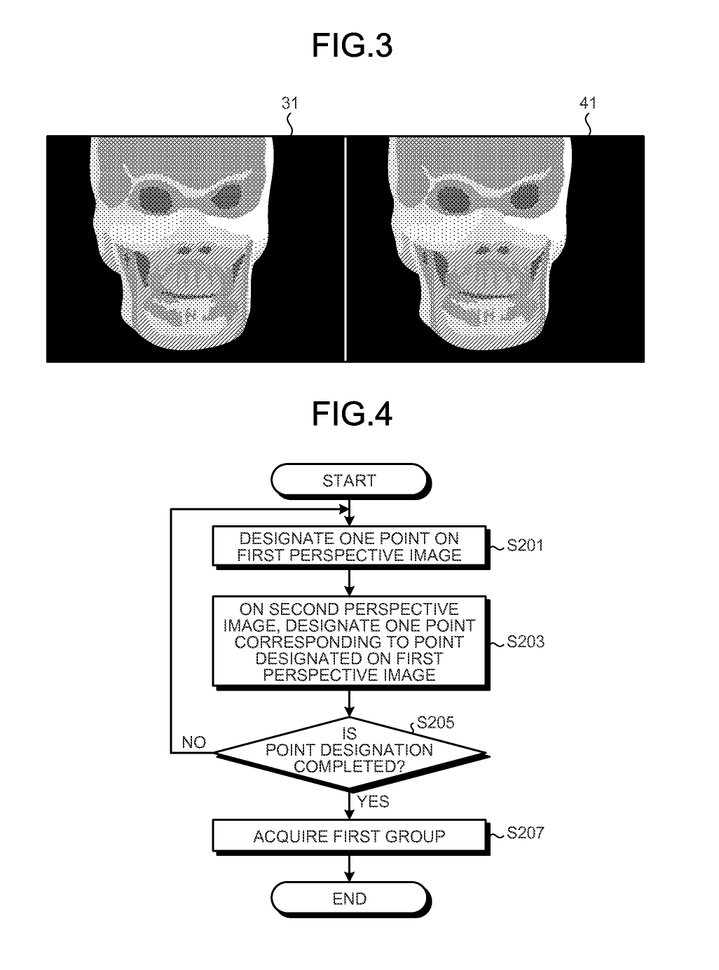

The display unit 15 then displays the first perspective image stored in the storage unit 11 and the second perspective image captured by the imaging unit 13 (Step S105). FIG. 3 is a schematic of an example of an arrangement of the first perspective image 31 and the second perspective image 41 according to the first embodiment. In the example illustrated in FIG. 3, the first perspective image 31 and the second perspective image 41 are displayed side by side, but may be displayed in any arrangement. Because the first perspective image 31 and the second perspective image 41 are fluoroscopic images of the subject captured from approximately the same directions, the resultant first perspective image 31 and second perspective image 41 are similar images, as illustrated in FIG. 3.

The first acquiring unit 17 then performs a corresponding point group acquiring process (Step S107). FIG. 4 is a flowchart of an example of the corresponding point group acquiring process according to the first embodiment.

The first acquiring unit 17 receives a designation of a point _pᵢ⁽¹⁾=(_uᵢ⁽¹⁾, _vᵢ⁽¹⁾) on the first perspective image displayed on the display unit 15 from the operator of the medical treatment apparatus 10 (Step S201). Here, i=1, . . . , N; N≧5; and _pᵢ⁽¹⁾ a two-dimensional coordinate point.

The first acquiring unit 17 then receives a designation of a corresponding point _pᵢ⁽²⁾=(_uᵢ⁽²⁾, _vᵢ⁽²⁾) corresponding to the point _pᵢ⁽¹⁾ designated on the first perspective image, on the second perspective image displayed on the display unit 15 from the operator of the medical treatment apparatus 10 (Step S203). Here, _pᵢ⁽²⁾ is a two-dimensional coordinate point.

If another pair of points is designated (No at Step S205), the value i is incremented, and the process from Step S201 to S203 is repeated. Pairs of points are kept being designated until designations of five or more pairs of corresponding points are completed, that is, until when i reaches a value equal to or more than five. The pair of _pᵢ⁽¹⁾, and _pᵢ⁽²⁾ represents a pair of corresponding points.

If the operator of the medical treatment apparatus 10 completes designations of the points (Yes at Step S205), the first acquiring unit 17 acquires a first group _P12={(p₁⁽¹⁾, _p₁⁽²⁾), . . . , (_p_{N}⁽¹⁾, _p_{N}⁽²⁾)} that includes the designated five or more pairs of corresponding points (Step S207).

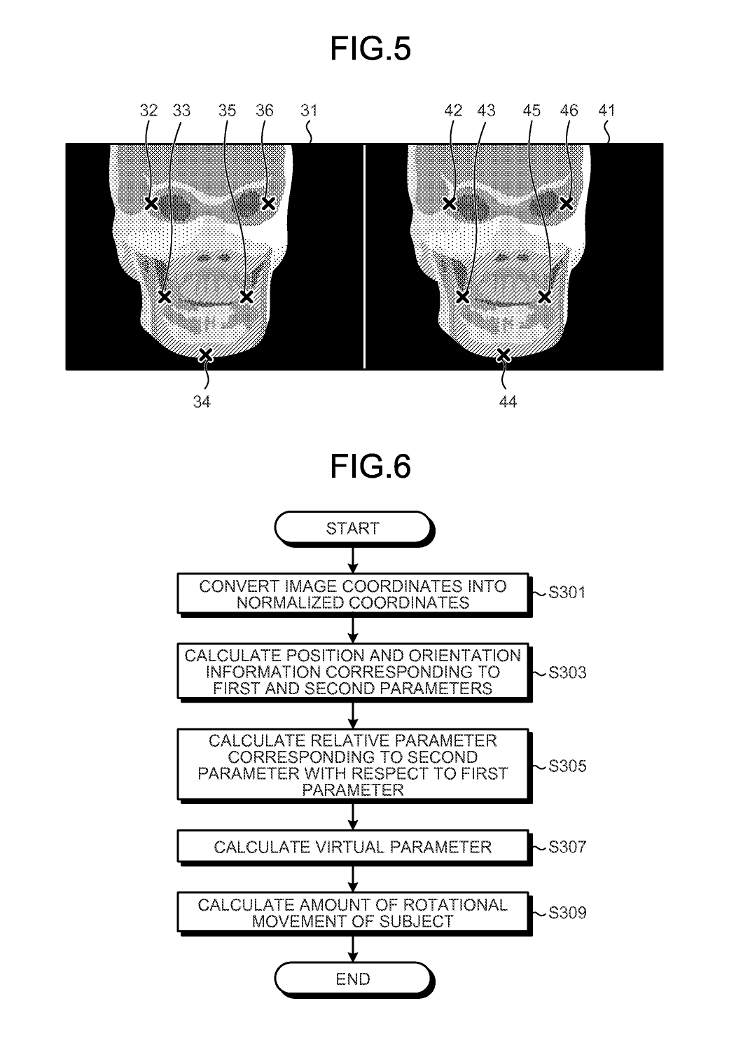

FIG. 5 is a schematic of an example of the first group _P12 according to the first embodiment. In the example illustrated in FIG. 5, a point 32 and a point 42, a point 33 and a point 43, a point 34 and a point 44, a point 35 and a point 45, and a point 36 and a point 46 are five pairs of corresponding points included in the first group _P12. In this manner, in the first embodiment, because the operator is allowed to designate these pairs of corresponding points on similar images, each of these pairs includes less error between the positions of the corresponding points.

In the example illustrated in FIG. 4, the first acquiring unit 17 may receive a designation of a point on the second perspective image at Step S201, and receive a designation of the corresponding point on the first perspective image at Step S203.

Referring back to FIG. 2, the calculating unit 21 then performs a displacement calculating process (Step S109). Before performing the displacement calculating process, the second acquiring unit 19 acquires the first parameter _C₁ from the storage unit 11, and acquires the second parameter _C₂ from the imaging unit 13. The first parameter _C₁ and the second parameter _C₂ are expressed as Equation (1).

_{—}Cⱼ=Aⱼ└Rⱼtⱼ┘ (1)

In Equation (1), j=1, 2; Aⱼ is a 3×3 conversion matrix for converting the physical coordinates into the image coordinates; Rⱼ is a 3×3 rotation matrix; and tⱼ is a 3×1 translation vector. Aⱼ is an example of the conversion information, and Rⱼ and tⱼ are examples of the position and orientation information.

The second acquiring unit 19 does not need to acquire the first parameter _C₁ and the second parameter _C₂ in their original format. The second acquiring unit 19 may acquire elements of the first parameter _C₁ and the second parameter _C₂, and may calculate the first parameter _C₁ and the second parameter _C₂ from the acquired elements.

It is generally known that _Cⱼ can be broken down into Aⱼ, Rⱼ, and tⱼ. Hence, the second acquiring unit 19 may acquire Aⱼ, Rⱼ, and tⱼ and calculate _Cⱼ for each of the first parameter and the second parameter, as an example. As another example, the second acquiring unit 19 may acquire Aⱼ, and [Rⱼ tⱼ] and calculate _Cⱼ for each of the first parameter and the second parameter. As another example, the second acquiring unit 19 may acquire elements of Aⱼ, Rⱼ, and tⱼ, and calculate _Cⱼ for each of the first parameter and the second parameter.

FIG. 6 is a flowchart of an example of the displacement calculating process according to the first embodiment. As described earlier, in the first embodiment, the amount of rotational movement (specifically, a rotation matrix) is calculated as the difference.

To begin with, the calculating unit 21 converts the first group P12 represented by the image coordinates into a first group P12={(p₁⁽¹⁾, p₁⁽²⁾), . . . , (p_{N}⁽¹⁾, p_{N}⁽²⁾)}, where pᵢ^{(j)}=(uᵢ^{(j)}, vᵢ^{(j)}), represented in the normalized coordinate system (Step S301). For example, the calculating unit 21 breaks down _Cⱼ into Aⱼ and [Rⱼ tⱼ], and converts the first group _P12 in the image coordinate system into the first group P12 in the normalized coordinate system using Equation (2).

$$[ \begin{matrix}{u}_{i}^{( j )} \\ {v}_{i}^{( j )} \\ 1\end{matrix} ] = {A}_{j}^{- 1} [ \begin{matrix}{}_{-}^{}{u}{}_{i}^{( j )} \\ {}_{-}^{}{v}{}_{i}^{( j )} \\ 1\end{matrix} ] \qquad ( 2 )$$

In Equation (2), Aⱼ⁻¹ is the inverse matrix of Aⱼ.

The calculating unit 21 then calculates the position and orientation information C₁ corresponding to the first parameter _C₁ and the position and orientation information C₂ corresponding to the second parameter _C₂ (Step S303). The calculating unit 21 calculates the position and orientation information C₁ and the position and orientation information C₂ using Equation (3), for example.

Cⱼ=A⁻¹_{—}Cⱼ=└Rⱼtⱼ┘ (3)

As expressed in Equation (3), the position and orientation information C₁ is a parameter based on the position and orientation of the imaging device (specifically, radioactive beam radiation unit) having captured the first perspective image at the first point in time, and the position and orientation information C₂ is a parameter based on the position and orientation of the imaging unit 13 (specifically, radioactive beam radiation unit) having captured the second perspective image at the second point in time.

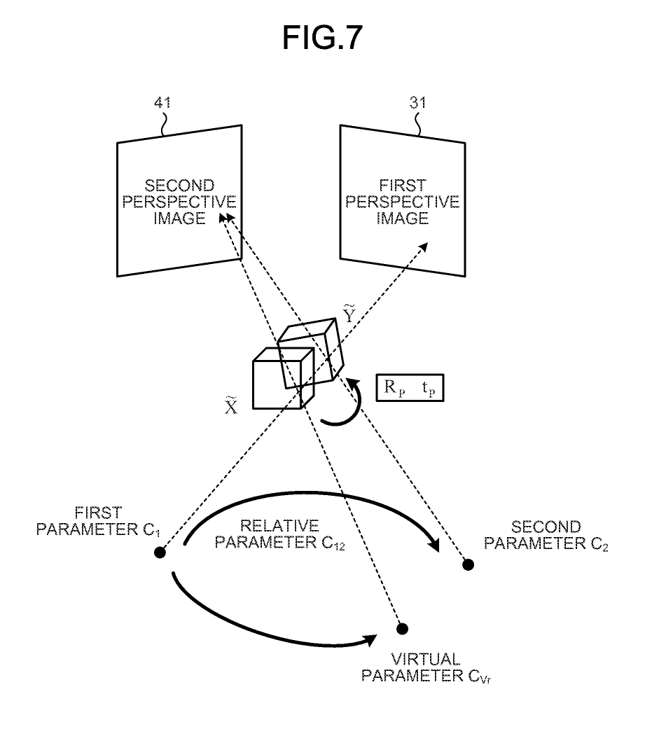

When X(i)=(x(i)₁, x(i)₂, x(i)₃) is the position of the i-th point in the subject in the actual space at the first point in time, and Y(i)=(y(i)₁, y(i)₂, y(i)₃) is the position of the i-th point in the subject corresponding to X(i) in the actual space at the second point in time, and when [Rₚ tₚ] is a conversion matrix for converting coordinates of a subject position in the actual space at the first point in time into coordinates of the subject position in the actual space at the second point in time (see FIG. 7), the relation between X(i) and Y(i) can be expressed as Equation (4). In FIG. 7, X˜ denotes the position of the subject at the first point in time, and Y˜ denotes the position of the subject at the second point in time.

$$[ \begin{matrix}{y ( i )}_{1} \\ {y ( i )}_{2} \\ {y ( i )}_{3} \\ 1\end{matrix} ] = [ \begin{matrix}{R}_{p} & {t}_{p} \\ 0 & 1\end{matrix} ] [ \begin{matrix}{x ( i )}_{1} \\ {x ( i )}_{2} \\ {x ( i )}_{3} \\ 1\end{matrix} ] \qquad ( 4 )$$

Here, Rₚ is a 3×3 rotation matrix, and t₂ is a 3×1 vector.

X(i) is projected on pᵢ⁽¹⁾=(uᵢ⁽¹⁾, vᵢ⁽¹⁾) with the position and orientation information C₁, and Y(i) is projected on pᵢ⁽²⁾=(uᵢ⁽²⁾, vᵢ⁽²⁾) with the position and orientation information C₂. The projection of X(i) onto pᵢ⁽¹⁾ is expressed by Equation (5), and the projection of Y(i) onto pᵢ⁽²⁾ is expressed by Equation (6).

$$\begin{matrix}{λ}_{i}^{( 1 )} [ \begin{matrix}{u}_{i}^{( 1 )} \\ {v}_{i}^{( 1 )} \\ 1\end{matrix} ] = [ \begin{matrix}{R}_{1} & {t}_{1}\end{matrix} ] [ \begin{matrix}{x ( i )}_{1} \\ {x ( i )}_{2} \\ {x ( i )}_{3} \\ 1\end{matrix} ] & ( 5 ) \\ {λ}_{i}^{( 2 )} [ \begin{matrix}{u}_{i}^{( 2 )} \\ {v}_{i}^{( 2 )} \\ 1\end{matrix} ] = [ \begin{matrix}{R}_{2} & {t}_{2}\end{matrix} ] [ \begin{matrix}{y ( i )}_{1} \\ {y ( i )}_{2} \\ {y ( i )}_{3} \\ 1\end{matrix} ] & ( 6 )\end{matrix}$$

Here, λᵢ⁽¹⁾ represents the third element in the three-dimensional vector resulting from the multiplication on the right side of Equation (5), and λᵢ⁽²⁾ represents the third element of the three-dimensional vector resulting from the multiplication on the right side of Equation (6).

The calculating unit 21 then calculates a relative parameter corresponding to the second parameter _C₂ with respect to the first parameter _C₁ (Step S305). Specifically, the calculating unit 21 calculates a relative parameter C₁₂ corresponding to the position and orientation information C₂ with respect to the position and orientation information C₁ (see FIG. 7).

For example, the calculating unit 21 expresses the relation between the position and orientation information C₁ and the position and orientation information C₂ as Equation (7).

$$[ \begin{matrix}{R}_{2} & {t}_{2} \\ 0 & 1\end{matrix} ] = [ \begin{matrix}{R}_{12} & {t}_{12} \\ 0 & 1\end{matrix} ] [ \begin{matrix}{R}_{1} & {t}_{1} \\ 0 & 1\end{matrix} ] \qquad ( 7 )$$

In Equation (7), 0=[0, 0, 0].

The calculating unit 21 then calculates [R₁₂ t₁₂] in Equation (7) as the relative parameter C₁₂ given by Equation (8), using the position and orientation information C₁ and the position and orientation information C₂.

$$[ \begin{matrix}{R}_{12} & {t}_{12} \\ 0 & 1\end{matrix} ] = {[ \begin{matrix}{R}_{2} & {t}_{2} \\ 0 & 1\end{matrix} ] [ \begin{matrix}{R}_{1} & {t}_{1} \\ 0 & 1\end{matrix} ]}^{- 1} \qquad ( 8 )$$

Equation (5) can be considered as a perspective projection of X(i) having its coordinates converted with [R₁ t₁], as expressed by Equation (9). Equation (6) can be considered as X(i) applied with the coordinate conversion with [R₁ t₁] and then projected with a virtual parameter C_{Vr}=[R_{Vr} t_{Vr}] (see FIG. 7), as expressed by Equation (10). The virtual parameter C_{Vr} is a parameter assuming that the second perspective image is captured while the subject is at the same position as when the first perspective image is captured, as illustrated in FIG. 7.

$$\begin{matrix}{λ}_{i}^{( 1 )} [ \begin{matrix}{u}_{i}^{( 1 )} \\ {v}_{i}^{( 1 )} \\ 1\end{matrix} ] = [ \begin{matrix}I & 0\end{matrix} ] [ \begin{matrix}{R}_{1} & {t}_{1} \\ 0 & 1\end{matrix} ] [ \begin{matrix}{x ( i )}_{1} \\ {x ( i )}_{2} \\ {x ( i )}_{3} \\ 1\end{matrix} ] & ( 9 ) \\ {λ}_{i}^{( 2 )} [ \begin{matrix}{u}_{i}^{( 2 )} \\ {v}_{i}^{( 2 )} \\ 1\end{matrix} ] = [ \begin{matrix}{R}_{Vr} & {t}_{Vr}\end{matrix} ] [ \begin{matrix}{R}_{1} & {t}_{1} \\ 0 & 1\end{matrix} ] [ \begin{matrix}{x ( i )}_{1} \\ {x ( i )}_{2} \\ {x ( i )}_{3} \\ 1\end{matrix} ] & ( 10 )\end{matrix}$$

The calculating unit 21 then calculates the virtual parameter C_{Vr}=[R_{Vr} t_{Vr}] (Step S307). The calculating unit 21 calculates the virtual parameter C_{Vr} with the five-point algorithm that is based on the epipolar geometry, for example. The five-point algorithm that is based on the epipolar geometry is disclosed in Kukelova, Zuzana, Martin Bujnak, and Tomas Pajdla, “Polynomial eigenvalue solutions to the 5-pt and 6-pt relative pose problems”, BMVC 2008 2.5 (2008), for example.

To explain specifically, a pair of corresponding points on the first perspective image and the second perspective image satisfies a relation expressed by Equation (11).

$${[ \begin{matrix}{u}_{i}^{( 2 )} \\ {v}_{i}^{( 2 )} \\ 1\end{matrix} ]}^{T} E [ \begin{matrix}{u}_{i}^{( 1 )} \\ {v}_{i}^{( 1 )} \\ 1\end{matrix} ] = 0 \qquad ( 11 )$$

Here, E is a fundamental matrix with three rows by three columns expressed by Equation (12), and the calculating unit 21 can obtain R_{Vr} by breaking down E. For t_{Vr}, the orientation of the vector can be calculated while keeping the scale of the vector undetermined. In the first embodiment, because the amount of rotational movement is used as the difference, as mentioned earlier, a calculation of R_{Vr} will be now explained assuming that t_{Vr} is a null vector with a three row and one column.

$$E = [ \begin{matrix}{E}_{11} & {E}_{12} & {E}_{13} \\ {E}_{21} & {E}_{22} & {E}_{23} \\ {E}_{31} & {E}_{32} & {E}_{33}\end{matrix} ] \qquad ( 12 )$$

The calculating unit 21 starts by selecting five pairs from the five or more pairs of corresponding points (pᵢ⁽¹⁾ and pᵢ⁽²⁾) in the first perspective image and the second perspective image. For example, the calculating unit 21 selects the five pairs satisfying i=1 to 5, or select the five pairs using RANdom Sample Consensus (RANSAC) algorithm, which is a robust estimation technique, for example.

The calculating unit 21 then creates a vector αi (α₁ to α₅) expressed by Equation (13) for each of the selected five pairs, and creates a vector E^{s} expressed by Equation (14) from the matrix E.

αᵢ≡└uᵢ⁽¹⁾uᵢ⁽²⁾vᵢ⁽¹⁾uᵢ⁽²⁾uᵢ⁽²⁾uᵢ⁽¹⁾vᵢ⁽²⁾vᵢ⁽¹⁾vᵢ⁽²⁾vᵢ⁽²⁾uᵢ⁽¹⁾vᵢ⁽¹⁾1┘ (13)

E^{s}≡└E₁₁E₁₂E₁₃E₂₁E₂₂E₂₃E₃₁E₃₂E₃₃┘^{T} (14)

The calculating unit 21 then defines a matrix B expressed by Equation (15) using created α1 to α5.

$$( 15 )$$

$$B = [ \begin{matrix}{a}_{1} ≡ [ \begin{matrix}{u}_{1}^{( 1 )} {u}_{1}^{( 2 )} & {v}_{1}^{( 1 )} {u}_{1}^{( 2 )} & {u}_{1}^{( 2 )} & {u}_{1}^{( 1 )} {v}_{1}^{( 2 )} & {v}_{1}^{( 1 )} {v}_{1}^{( 2 )} & {v}_{1}^{( 2 )} & {u}_{1}^{( 1 )} & {v}_{1}^{( 1 )} & 1\end{matrix} ] \\ {a}_{2} ≡ [ \begin{matrix}{u}_{2}^{( 1 )} {u}_{2}^{( 2 )} & {v}_{2}^{( 1 )} {u}_{2}^{( 2 )} & {u}_{2}^{( 2 )} & {u}_{2}^{( 1 )} {v}_{2}^{( 2 )} & {v}_{2}^{( 1 )} {v}_{2}^{( 2 )} & {v}_{2}^{( 2 )} & {u}_{2}^{( 1 )} & {v}_{2}^{( 1 )} & 1\end{matrix} ] \\ {a}_{3} ≡ [ \begin{matrix}{u}_{3}^{( 1 )} {u}_{3}^{( 2 )} & {v}_{3}^{( 1 )} {u}_{3}^{( 2 )} & {u}_{3}^{( 2 )} & {u}_{3}^{( 1 )} {v}_{3}^{( 2 )} & {v}_{3}^{( 1 )} {v}_{3}^{( 2 )} & {v}_{3}^{( 2 )} & {u}_{3}^{( 1 )} & {v}_{3}^{( 1 )} & 1\end{matrix} ] \\ {a}_{4} ≡ [ \begin{matrix}{u}_{4}^{( 1 )} {u}_{4}^{( 2 )} & {v}_{4}^{( 1 )} {u}_{4}^{( 2 )} & {u}_{4}^{( 2 )} & {u}_{4}^{( 1 )} {v}_{4}^{( 2 )} & {v}_{4}^{( 1 )} {v}_{4}^{( 2 )} & {v}_{4}^{( 2 )} & {u}_{4}^{( 1 )} & {v}_{4}^{( 1 )} & 1\end{matrix} ] \\ {a}_{5} ≡ [ \begin{matrix}{u}_{5}^{( 1 )} {u}_{5}^{( 2 )} & {v}_{5}^{( 1 )} {u}_{5}^{( 2 )} & {u}_{5}^{( 2 )} & {u}_{5}^{( 1 )} {v}_{5}^{( 2 )} & {v}_{5}^{( 1 )} {v}_{5}^{( 2 )} & {v}_{5}^{( 2 )} & {u}_{5}^{( 1 )} & {v}_{5}^{( 1 )} & 1\end{matrix} ]\end{matrix} ]$$

Between the matrix B and the vector E^{s}, the relation expressed by Equation (16) is established.

BE^{s}=0 (16)

Therefore, by performing a singular value decomposition of the matrix B and acquiring four bases satisfying Equation (16), the calculating unit 21 can express the vector E^{s} by a linear combination of four bases e₁ to e₄, as expressed by Equation (17).

E^{s}=le₁+me₂+ne₃+e₄ (17)

The matrix E satisfies Equations (18) and (19)

det(E)=0 (18)

2EE^{T}E−trace(EE^{T})E=0 (19)

Therefore, the relation expressed by Equation (20) is established based on Equations (17) to (19).

Wα=0 (20)

Here, W is a matrix with 10 rows and 20 columns, and α is a twenty-dimensional vector defined by Equation (21).

α=(l³,ml²,m²l,m³,nl²,nml,nm²,n²l,n²m,n³,l²,ml,m²,nl,nm,n²,l,m,n,1)^{T} (21)

Hence, Equation (20) can be transformed as expressed by Equation (22).

(n³F₃+n²F₂+nF₁+F₀)β=0 (22)

Here, β is a ten-dimensional vector defined by Equation (23).

β=(l³l²mlm²m³l²lmm²l,m1)^{T} (23)

In Equation (22), F₃ to F₀ are matrixes each with ten rows and ten columns defined by Equations (24) to (27), respectively.

F₃≡(000000000w₁₀) (24)

F₂≡(0000000w₈w₉w₁₆) (25)

F₁≡(0000w₅w₆w₇w₁₄w₁₅w₁₉) (26)

F₀≡(w₁w₂w₃w₄w₁₁w₁₂w₁₃w₁₇w₁₈w₂₀) (27)

Here, 0 is a ten-dimensional column vector, and wₙ is a column vector at the n-th column of the matrix W.

Equation (22) is an eigenvalue problem involving a cubic polynomial, and the calculating unit 21 can find n and β using known algorithms. An example of the known algorithm is MATLAB's polyeig function. The calculating unit 21 can then find l and m from found β and Equation (23). This process yields a plurality of solutions for l, m, and n, and the fundamental matrix E, which is given by Equation (17), in plurality as well. Hereinafter, these fundamental matrixes are expressed as E_{q} (q≧1). The calculating unit 21 breaks down the fundamental matrixes E, and acquires a plurality of candidates for R_{Vr}. Hereinafter, these candidates for R_{Vr} are also denoted by E_{Vr}.

The calculating unit 21 then calculates the amount of rotational movement of the subject from the candidates for R_{Vr} (Step S309).

The calculating unit 21 starts by transforming Equation (6) into Equation (28) using Equations (4) and (7).

$$\begin{matrix}{λ}_{i}^{( 2 )} [ \begin{matrix}{u}_{i}^{( 2 )} \\ {v}_{i}^{( 2 )} \\ 1\end{matrix} ] = [ \begin{matrix}{R}_{2} & {t}_{2}\end{matrix} ] [ \begin{matrix}{y ( i )}_{1} \\ {y ( i )}_{2} \\ {y ( i )}_{3} \\ 1\end{matrix} ] \\ = [ \begin{matrix}{R}_{12} & {t}_{12}\end{matrix} ] [ \begin{matrix}{R}_{1} & {t}_{1} \\ 0 & 1\end{matrix} ] [ \begin{matrix}{R}_{p} & {t}_{p} \\ 0 & 1\end{matrix} ] [ \begin{matrix}{x ( i )}_{1} \\ {x ( i )}_{2} \\ {x ( i )}_{3} \\ 1\end{matrix} ]\end{matrix} \qquad ( 28 )$$

The relation expressed by Equation (29) is established based on Equations (10) and (28).

$$[ \begin{matrix}{R}_{Vr} & {t}_{Vr} \\ 0 & 1\end{matrix} ] [ \begin{matrix}{R}_{1} & {t}_{1} \\ 0 & 1\end{matrix} ] = [ \begin{matrix}{R}_{12} & {t}_{12} \\ 0 & 1\end{matrix} ] [ \begin{matrix}{R}_{1} & {t}_{1} \\ 0 & 1\end{matrix} ] [ \begin{matrix}{R}_{p} & {t}_{p} \\ 0 & 1\end{matrix} ] \qquad ( 29 )$$

Here, the conversion matrix [Rₚ tₚ] can be calculated from Equation (30) based on Equation (29).

$$[ \begin{matrix}{R}_{p} & {t}_{p} \\ 0 & 1\end{matrix} ] = {{[ \begin{matrix}{R}_{1} & {t}_{1} \\ 0 & 1\end{matrix} ]}^{- 1} [ \begin{matrix}{R}_{12} & {t}_{12} \\ 0 & 1\end{matrix} ]}^{- 1} [ \begin{matrix}{R}_{Vr} & {t}_{Vr} \\ 0 & 1\end{matrix} ] [ \begin{matrix}{R}_{1} & {t}_{1} \\ 0 & 1\end{matrix} ] \qquad ( 30 )$$

The calculating unit 21 calculates the rotational angles (ΔRₓ, ΔR_{y}, ΔR_{z}) about the X axis, the Y axis, and the Z axis in the actual space from the amount of rotational movement Rₚ of the subject, where Rₚ is a matrix expressed by Equation (31).

$${R}_{p} = [ \begin{matrix}{r}_{{p}_{1}} & {r}_{{p}_{2}} & {r}_{{p}_{3}} \\ {r}_{{p}_{4}} & {r}_{{p}_{5}} & {r}_{{p}_{6}} \\ {r}_{{p}_{7}} & {r}_{{p}_{8}} & {r}_{{p}_{9}}\end{matrix} ] \qquad ( 31 )$$

For example, the calculating unit 21 uses Equations (32) to (34) to calculate the rotational angles (ΔRₓ, ΔR_{y}, ΔR_{z}) about the X axis, the Y axis, and the Z axis.

ΔRₓ=sin⁻¹(rₚ₄/\sqrt{1−rₚ₇²}) (32)

ΔE_{y}=sin⁻¹(−rₚ₇) (33)

ΔR_{z}=sin⁻¹(rₚ₈/\sqrt{1−rₚ₇²}) (34)

In this example, it is assumed that the rotations about the X axis, the Y axis, and the Z axis occur in the order listed herein. In the first embodiment, it can also be assumed that the amount of rotation of the subject at the second point in time with respect to the position of the subject at the first point in time is small. Based on these assumptions, the calculating unit 21 calculates the rotational angles (ΔRₓ, ΔR_{y}, ΔR_{z}) for each of the candidates of R_{Vr} using Equations (30) to (34), and selects a set of rotational angles whose sum (ΔRₓ+ΔR_{y}+ΔR_{z}) is the smallest from those having calculated, as the amount of rotational movement of the subject.

When the appropriate five pairs are selected from the first group with RANSAC a plurality of number of times and the displacement calculating process is performed a plurality of number of times, the calculating unit 21 can acquire one of the amounts of rotational movement, each of which is calculated every time the displacement calculating process is performed, whose sum of the rotational angles (ΔRₓ+ΔR_{y}+ΔR_{z}) is the smallest, as the final amount of rotational movement of the subject.

Referring back to FIG. 2, the control unit 23 controls to adjust the position of subject to the position of the subject at the first point in time by rotating the bed on which the subject lies (not illustrated), using the difference (amount of rotation) calculated by the calculating unit 21 (Step S111).

In the first embodiment, the bed can be rotated about the X axis, the Y axis, and the Z axis. The control unit 23 rotates the bed about the Z axis by ΔR_{z}, rotates the bed about the Y axis by ΔR_{y}, and finally rotates the bed about the X axis by ΔRₓ, based on the difference (amounts of rotation) calculated by the calculating unit 21.

The radiation unit 25 then irradiates a therapeutic beam to the affected area of the subject of which the position has been controlled, in accordance with the irradiation information set by the operator of the medical treatment apparatus 10 during the treatment planning (Step S113).

In this way, according to the first embodiment, operators can designate pairs of corresponding points on similar images. In this manner, designation error between the corresponding points in each pair can be reduced, so that the accuracy of the subject positioning control can be improved. Furthermore, according to the first embodiment, operators of the medical treatment apparatus 10 can easily designate pairs of corresponding points, so that burdens of the operators can be reduced.

### Second Embodiment

Explained in a second embodiment is an example in which the amount of rotational movement and the amount of translational movement of the subject from the first point in time to the second point in time are calculated, and the position of the subject is controlled using the calculated amounts of rotational movement and translational movement before conducting the radiotherapy.

According to the second embodiment, when a displacement of the subject from the first point in time to the second point in time results from rotational movement and translational movement, the amount of rotational movement and the amount of translational movement causing the displacement can be corrected before the radiotherapy is conducted.

The explanation hereunder will focus on differences with the first embodiment, and elements having the same functions as those in the first embodiment will be given the same name and reference numerals as those in the first embodiment, and explanations thereof will be omitted herein.

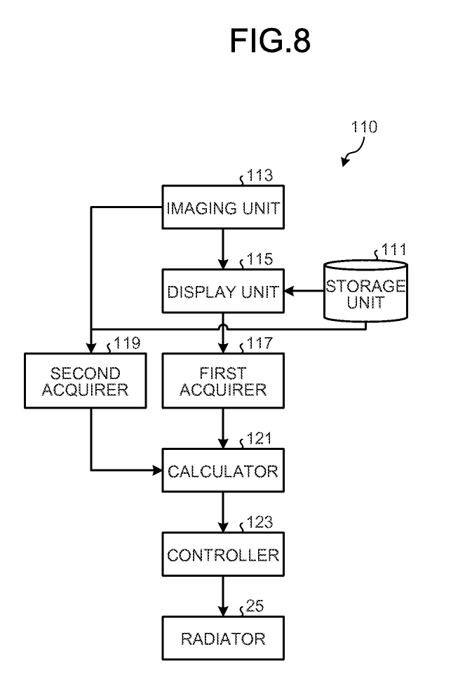

FIG. 8 is a schematic illustrating an example of a configuration of a medical treatment apparatus 110 according to the second embodiment. As illustrated in FIG. 8, in the medical treatment apparatus 110 according to the second embodiment, a storage unit 111, an imaging unit 113, a display unit 115, a first acquiring unit 117, a second acquiring unit 119, a calculating unit 121, and a control unit 123 are different from those according to the first embodiment.

Additional information stored in the storage unit 111 includes a third perspective image that is a fluoroscopic image of a subject captured at the first point in time from a second direction that is different from the first direction, position and orientation information related to the position and orientation of the imaging device capturing the third perspective image at the first point in time, a third parameter including conversion information related to conversions of the normalized coordinate system into a third perspective image coordinate system.

In the second embodiment, the imaging device capturing the third perspective image is considered to be an imaging unit (not illustrated) that is not the imaging unit 113, which is explained later, but may be the imaging unit 113. Specifically, the imaging device capturing the third perspective image is the same imaging device capturing the first perspective image, but the third perspective image is captured by a radioactive beam radiation unit and a sensor that are not those used in capturing the first perspective image. The third parameter can be implemented as a camera parameter (external parameter and internal parameter) of the imaging device capturing the third perspective image (more specifically, the radioactive beam radiation unit for capturing the third perspective image) at the first point in time, and can be acquired through calibration of the imaging device capturing the third perspective image. The imaging device capturing the third perspective image may also be implemented as a CT device, in the same manner as the imaging device used in the first embodiment.

The imaging unit 113 further captures a fourth perspective image that is a fluoroscopic image of the subject captured at the second point in time from a direction approximately the same as the second direction. In the second embodiment, the same direction as the second direction is assumed as the direction approximately the same as the second direction, but some error is tolerable. In other words, one of the directions centered about the second direction within a certain error can be used as the direction approximately the same as the second direction. The certain error may be predetermined, or may be visually determined by an operator (radiographer) of the medical treatment apparatus 110 such as a physician. The fourth perspective image is captured by a radioactive beam radiation unit and a sensor that are not those used in capturing the second perspective image by the imaging unit 113.

The display unit 115 displays the first perspective image and the third perspective image stored in the storage unit 111, and the second perspective image and the fourth perspective image captured by the imaging unit 113.

The first acquiring unit 117 further acquires a second group that includes one or more pairs of corresponding points between the third perspective image and the fourth perspective image, such one or pairs corresponding to at least one of the pairs in the first group. The second group has one or more pairs of corresponding points corresponding to at least a pair of corresponding points in the first group, and designated on the third perspective image and the fourth perspective image displayed on the display unit 115. In other words, the first acquiring unit 117 acquires one or more pairs of corresponding points corresponding to at least one of the pairs in the first group, such one or more pairs being designated on the third perspective image and the fourth perspective image displayed on the display unit 115, as the second group. The one or more pairs of corresponding points on the third perspective image and the fourth perspective image are designated in the same manner as in the first embodiment.

The second acquiring unit 119 further acquires the third parameter, position and orientation information related to the position and orientation of the imaging unit 113 capturing the fourth perspective image at the second point in time, and a fourth parameter including conversion information related to conversions of the normalized coordinate system into a fourth perspective image coordinate system. The fourth parameter can be implemented as a camera parameter (an external parameter or an internal parameter) of the imaging unit 113 (specifically, the radioactive beam radiation unit capturing the fourth perspective image) at the second point in time, for example, and can be acquired through calibration of the imaging unit 113 for capturing the fourth perspective image. Specifically, the second acquiring unit 119 further acquires the third parameter from the storage unit 111, and further acquires the fourth parameter from the imaging unit 113.

The calculating unit 121 further calculates the amount of translational movement of the subject from the first point in time to the second point in time using the second group acquired by the first acquiring unit 117, and the third parameter and the fourth parameter acquired by the second acquiring unit 119. In the second embodiment, the difference corresponds to the amount of translational movement as well as the amount of rotational movement of the subject from the first point in time to the second point in time (more specifically, the amount of rotational movement and the amount of translational movement of the subject at the second point in time with respect to the position of the subject at the first point in time).

The control unit 123 controls to adjust the position of the subject to the position of the subject at the first point in time using the amount of rotational movement and the amount of translational movement calculated by the calculating unit 121. To control to adjust the position of the subject to the position of the subject at the first point in time, the control unit 123 rotates the bed on which the subject lies (not illustrated) using the amount of rotational movement and the amount of translational movement calculated by the calculating unit 121.

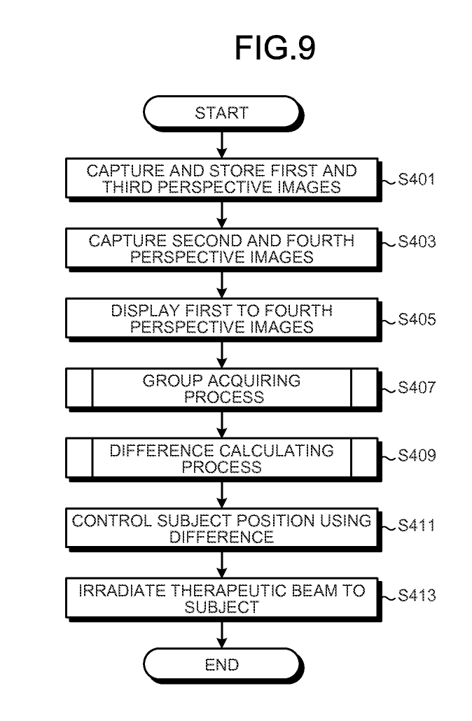

FIG. 9 is a flowchart of an example of a process performed by the medical treatment apparatus 110 according to the second embodiment.

To begin with, the imaging device for capturing the first perspective image and the third perspective image captures the first perspective image that is a fluoroscopic image of the subject from the first direction, and the third perspective image that is another fluoroscopic image of the subject from the second direction at the first point in time. The captured first perspective image, the first parameter of the imaging device capturing the first perspective image, the captured third perspective image, and the third parameter of the imaging device capturing the third perspective image are then stored in the storage unit 111 (Step S401).

At the second point in time, the imaging unit 113 captures the second perspective image that is a fluoroscopic image of the subject from a direction approximately the same as the first direction, and captures the fourth perspective image that is another fluoroscopic image of the subject from a direction approximately the same as the second direction (Step S403).

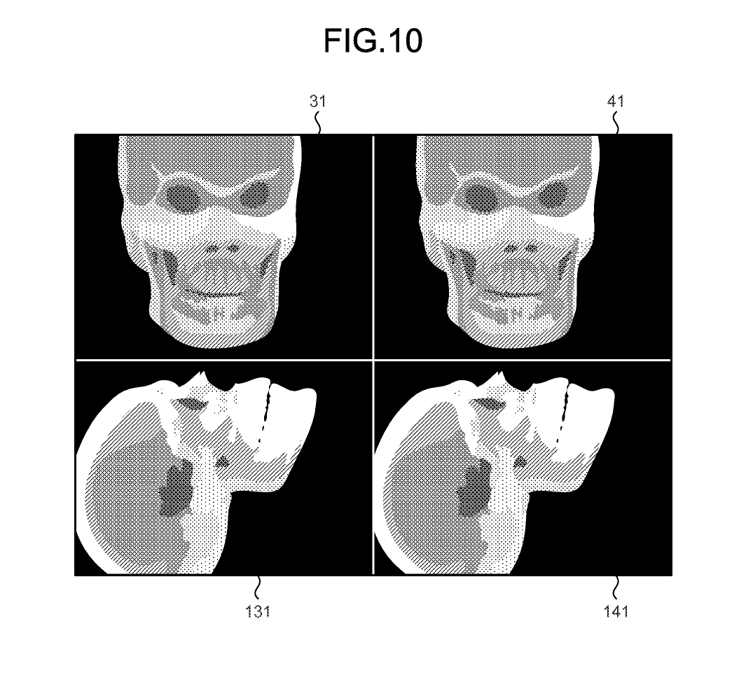

The display unit 115 then displays the first perspective image and the third perspective image stored in the storage unit 111, and the second perspective image and the fourth perspective image captured by the imaging unit 113 (Step S405). FIG. 10 is a schematic of an example of an arrangement of the first perspective image 31, the second perspective image 41, the third perspective image 131, and the fourth perspective image 141 according to the second embodiment. In the example illustrated in FIG. 10, the first perspective image 31 and the second perspective image 41 are displayed side by side, and the third perspective image 131 and the fourth perspective image 141 are displayed side by side under the first perspective image 31 and the second perspective image 41, but the first to fourth perspective images 31 to 141 may be displayed in any arrangement. As illustrated in FIG. 10, the first perspective image 31 and the second perspective image 41 are similar images because they are fluoroscopic images of the subject captured from approximately the same directions, and the third perspective image 131 and the fourth perspective image 141 are similar images because they are fluoroscopic images of the subject captured from approximately the same directions.

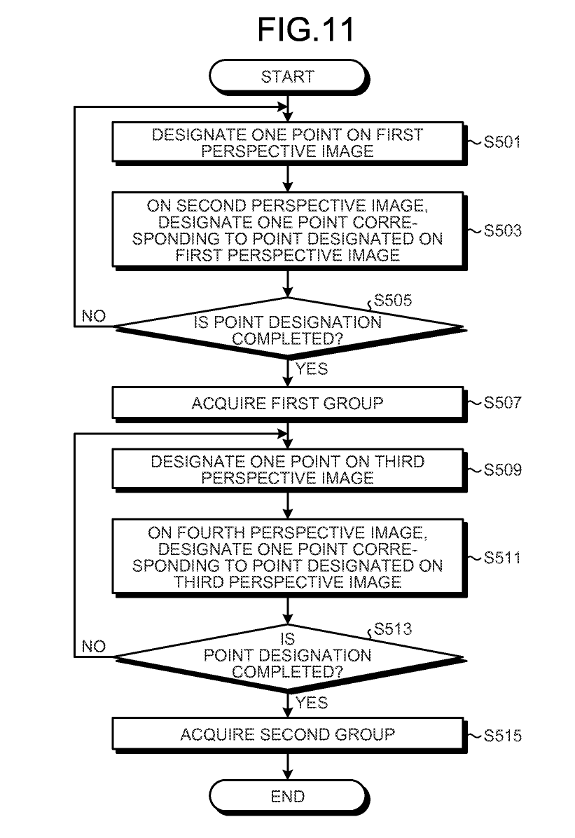

The first acquiring unit 117 then performs the corresponding point group acquiring process (Step S407). FIG. 11 is a flowchart of an example of the corresponding point group acquiring process according to the second embodiment.

Steps S501 to S507 are the same as Steps S201 to S207 illustrated in FIG. 4.

The first acquiring unit 117 receives a designation of a point _pₛ⁽³⁾=(_uₛ⁽³⁾, _vₛ⁽³⁾) on the third perspective image displayed on the display unit 115 from the operator of the medical treatment apparatus 110 (Step S509), where s=1, . . . , L, and L≧1. The point _pₛ⁽³⁾ corresponds to at least one of the points of the pairs in the first group. In the explanation of the second embodiment, L=1. _pₛ⁽³⁾ is a two-dimensional coordinate point.

The first acquiring unit 117 then receives a designation of the corresponding point _pₛ⁽⁴⁾=(_uₛ⁽⁴⁾, _vₛ⁽⁴⁾) corresponding to the point _pₛ⁽³⁾ designated on the third perspective image, on the fourth perspective image displayed on the display unit 115 from the operator of the medical treatment apparatus 110 (Step S511). _pₛ⁽⁴⁾ is a two-dimensional coordinate point.

If another pair of points is designated (No at Step S513), the value s is incremented, and the process from Steps S509 to S511 is repeated. Pairs of points are kept being designated until designations of one or more pairs of corresponding points are completed, that is, until when s reaches a value equal to or more than one. A pair of _pₛ⁽³⁾ and _pₛ⁽⁴⁾ represents a pairs of corresponding points.

If the operator of the medical treatment apparatus 110 completes designations of the points (Yes at Step S513), the first acquiring unit 117 acquires the second group _P34={(_p₁⁽³⁾, _p₁⁽⁴⁾), . . . , (_p_{L}⁽³⁾, _p_{L}⁽⁴⁾)} that includes the designated one or more pairs of corresponding points (Step S515).

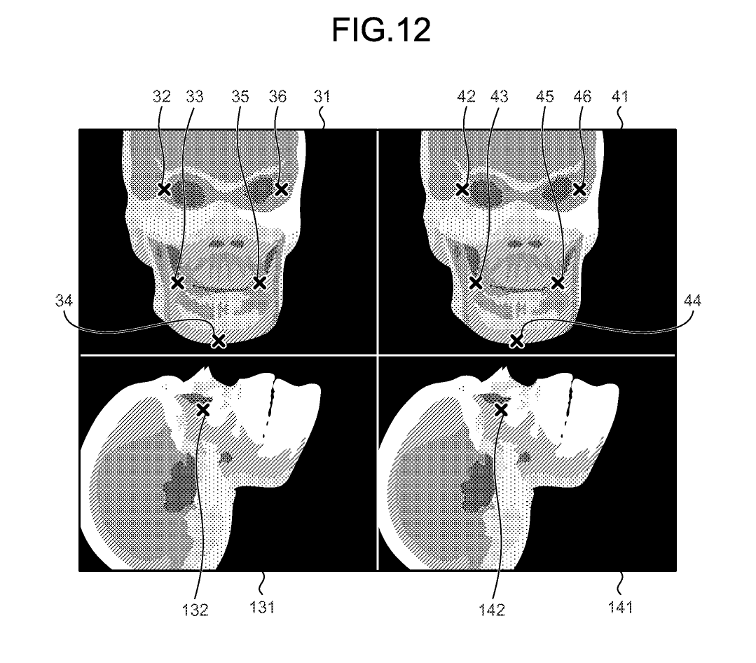

FIG. 12 is a schematic of an example of the first group _P12 and the second group _P34 according to the second embodiment. In the example illustrated in FIG. 12, the point 32 and the point 42, the point 33 and the point 43, the point 34 and the point 44, the point 35 and the point 45, and the point 36 and the point 46 are five pairs of corresponding points in the first group _P12. A point 132 and a point 142 corresponding to the pair of the point 32 and the point 42 are one pair of corresponding points in the second group _P34. In this manner, in the second embodiment, because designations of corresponding point pairs on dissimilar images are minimized, influence of error in designations of corresponding points in each pair can be reduced.

In the example illustrated in FIG. 11, the first acquiring unit 117 may receive a designation of one point corresponding to at least one of the points in the pairs in the first group on the fourth perspective image at Step S509, and receive a designation of the corresponding point on the third perspective image at Step S511.

Referring back to FIG. 9, the calculating unit 121 then performs the displacement calculating process (Step S409). Before performing the displacement calculating process, the second acquiring unit 119 acquires the first parameter _C₁ and the third parameter _C₃ from the storage unit 111, and acquires the second parameter _C₂ and the fourth parameter _C₄ from the imaging unit 113. The first parameter _C₁ to the fourth parameter _C₄ are expressed by Equation (35). The first parameter _C₁ to the fourth parameter _C₄ are acquired in the same manner as in the first embodiment.

$$- {C}^{( j )} = [ \begin{matrix}{c}_{11}^{( j )} & {c}_{12}^{( j )} & {c}_{13}^{( j )} & {c}_{14}^{( j )} \\ {c}_{21}^{( j )} & {c}_{22}^{( j )} & {c}_{23}^{( j )} & {c}_{24}^{( j )} \\ {c}_{31}^{( j )} & {c}_{32}^{( j )} & {c}_{33}^{( j )} & {c}_{34}^{( j )}\end{matrix} ] \qquad ( 35 )$$

Here, j is the parameter number, and takes a number from 1 to 4.

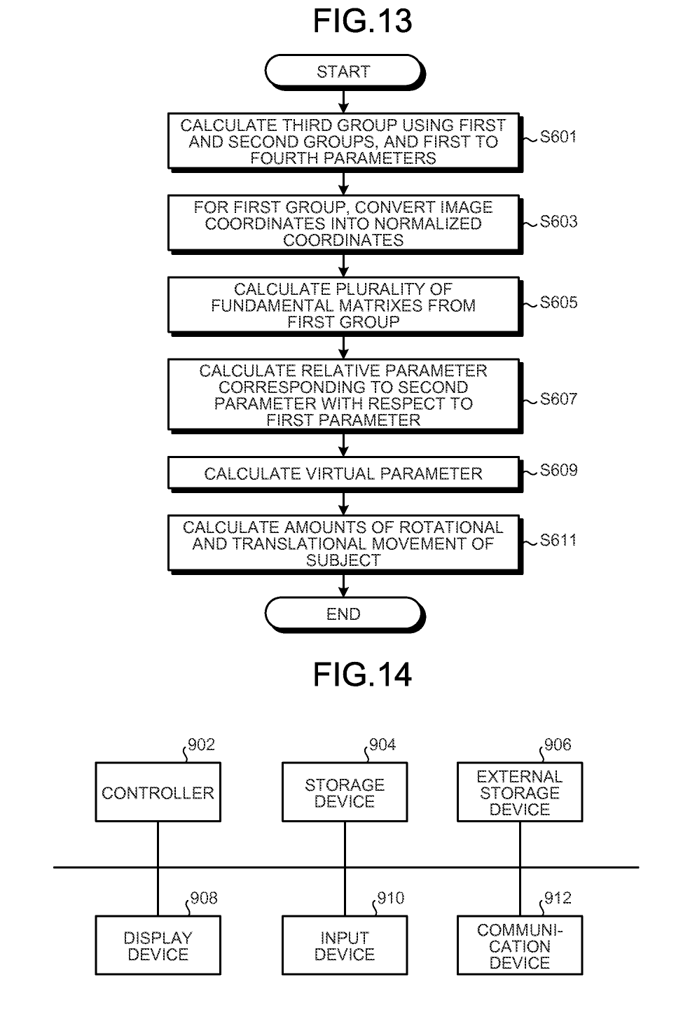

FIG. 13 is a flowchart of an example of the displacement calculating process according to the second embodiment. As mentioned earlier, in the second embodiment, the amount of rotational movement (specifically, rotation matrix) and the amount of translational movement (specifically, translation vector) are calculated as the difference.

To begin with, the calculating unit 121 calculates a third corresponding point group using the first group _P12, the second group _P34, the first parameter _C₁, the second parameter _C₂, the third parameter _C₃, and the fourth parameter _C₄ (Step S601).

For example, the calculating unit 121 acquires a corresponding point group _P1234 (_pₛ⁽¹⁾, _pₛ⁽²⁾, _pₛ⁽³⁾, _pₛ⁽⁴⁾), including the point pair corresponding to the pair in the second group _P34 among those in the first group _P12, and including the pair in the second group _P34. Here, _it is assumed that pₛ^{(j)}=(_uₛ^{(j)}, _vₛ^{(j)}).

The calculating unit 121 then calculates coordinates X(s)=(x(s)₁, x(s)₂, x(s)₃) in the actual space at the first point in time using (_pₛ⁽¹⁾, _pₛ⁽³⁾) from the corresponding point group _P1234 (_pₛ⁽¹⁾, _pₛ⁽²⁾, _pₛ⁽³⁾, _pₛ⁽⁴⁾), the first parameter _C₁, and the third parameter _C₃, as expressed by Equation (36).

$$[ \begin{matrix}{x ( s )}_{1} \\ {x ( s )}_{2} \\ {x ( s )}_{3}\end{matrix} ] = {[ \begin{matrix}{c}_{31}^{( 1 )} {u}_{s}^{( 1 )} - {c}_{11}^{( 1 )} & {c}_{32}^{( 1 )} {u}_{s}^{( 1 )} - {c}_{12}^{( 1 )} & {c}_{33}^{( 1 )} {u}_{s}^{( 1 )} - {c}_{13}^{( 1 )} \\ {c}_{31}^{( 1 )} {v}_{s}^{( 1 )} - {c}_{21}^{( 1 )} & {c}_{32}^{( 1 )} {v}_{s}^{( 1 )} - {c}_{22}^{( 1 )} & {c}_{33}^{( 1 )} {v}_{s}^{( 1 )} - {c}_{23}^{( 1 )} \\ {c}_{31}^{( 3 )} {u}_{s}^{( 3 )} - {c}_{11}^{( 3 )} & {c}_{32}^{( 3 )} {u}_{s}^{( 3 )} - {c}_{12}^{( 3 )} & {c}_{33}^{( 3 )} {u}_{s}^{( 3 )} - {c}_{13}^{( 3 )} \\ {c}_{31}^{( 3 )} {v}_{s}^{( 3 )} - {c}_{21}^{( 3 )} & {c}_{32}^{( 3 )} {v}_{s}^{( 3 )} - {c}_{22}^{( 3 )} & {c}_{33}^{( 3 )} {v}_{s}^{( 3 )} - {c}_{23}^{( 3 )}\end{matrix} ]}^{- 1} [ \begin{matrix}{c}_{14}^{( 1 )} - {c}_{34}^{( 1 )} {u}_{s}^{( 1 )} \\ {c}_{24}^{( 1 )} - {c}_{34}^{( 1 )} {u}_{s}^{( 1 )} \\ {c}_{14}^{( 3 )} - {c}_{34}^{( 3 )} {u}_{s}^{( 3 )} \\ {c}_{24}^{( 3 )} - {c}_{34}^{( 3 )} {u}_{s}^{( 3 )}\end{matrix} ] \qquad ( 36 )$$

Similarly, the calculating unit 121 calculates coordinates Y(s)=(y(s)₁, y(s)₂, y(s)₃) in the actual space at the second point in time using (_pₛ⁽²⁾, _pₛ⁽⁴⁾) from the corresponding point group _P1234 (_pₛ⁽¹⁾, _pₛ⁽²⁾, _pₛ⁽³⁾, _pₛ⁽⁴⁾), and the second parameter _C₂, and the fourth parameter _C₄.

Through this process, a third corresponding point group Q={(x(s)₁, x(s)₂, x(s)₃), (y(s)₁, y(s)₂, y(s)₃)} which are pairs of corresponding points at the first point in time and the second point in time in the actual space is acquired.

When the second group _P34 has two or more pairs of corresponding points, the calculating unit 121 may calculate two or more pairs of corresponding points in the actual space at a first point in time and the second point in time, and use one of the pairs as the third corresponding point group Q.

The calculating unit 121 then converts the first group _P12 in the image coordinates into a first group P12={(p₁⁽¹⁾, p₁⁽²⁾), . . . , (p_{N}⁽¹⁾, p_{N}⁽²⁾)} in the normalized coordinates, where pᵢ^{(j)}=(uᵢ^{(j)}, vᵢ^{(j)}) (Step S603). For example, the calculating unit 21 converts the first group _P12 in the image coordinates into the first group P12 in the normalized coordinates using Equation (2).

The calculating unit 121 then calculates a plurality of fundamental matrixes E_{q} (q≧1) using the first group P12 in the normalized coordinates (Step S605). Because the fundamental matrixes E_{q} are calculated in the same manner as in the first embodiment, the explanation thereof is omitted hereunder. Generally, a fundamental matrix can be broken down into a rotation matrix and a translation vector having a scale of one.

The calculating unit 121 then calculates a relative parameter corresponding to the second parameter _C₂ with respect to the first parameter _C₁ (Step S607). Specifically, the calculating unit 121 calculates a relative parameter C₁₂ corresponding to the position and orientation information C₂ with respect to the position and orientation information C₁. Because the relative parameter C₁₂ is calculated in the same manner as in the first embodiment, the explanation thereof is omitted herein.

The calculating unit 121 then calculates the virtual parameter C_{Vr}=[R_{Vr} t_{Vr}] (Step S609).

To explain specifically, the calculating unit 121 breaks down the fundamental matrixes E_{q} into a plurality of rotation matrixes R_{Vr} and a plurality of vectors tn_{Vr} each of which has information of the direction of t_{Vr} and is paired with the corresponding R_{Vr}.

Here, tn_{Vr} is a vector resulting from normalizing t_{Vr} to the scale of one (that is, |tn_{Vr}|=1); t_{Vr} can be expressed as t_{Vr}=αtn_{Vr}; and α is a scalar quantity representing the scale of tn_{Vr}. Hereinafter, a plurality of R_{Vr} and tn_{Vr} are represented as Rᵢ and tnᵢ, respectively.

The calculating unit 121 then acquires a desired R_{Vr} from Rᵢ and a desired tn_{Vr} from tnᵢ. Specifically, the calculating unit 121 calculates the difference of the subject (Rₚ, tₚ) with Equation (37), based on Equation (30).

$$\begin{matrix}[ \begin{matrix}{R}_{p} & {t}_{p} \\ 0 & 1\end{matrix} ] = {{[ \begin{matrix}{R}_{1} & {t}_{1} \\ 0 & 1\end{matrix} ]}^{- 1} [ \begin{matrix}{R}_{12} & {t}_{12} \\ 0 & 1\end{matrix} ]}^{- 1} [ \begin{matrix}{R}_{i} & α t {n}_{i} \\ 0 & 1\end{matrix} ] [ \begin{matrix}{R}_{1} & {t}_{1} \\ 0 & 1\end{matrix} ] \\ = {[ \begin{matrix}{R}_{1}^{- 1} & - {R}_{1}^{- 1} {t}_{1} \\ 0 & 1\end{matrix} ] [ \begin{matrix}{R}_{12}^{- 1} & - {R}_{12}^{- 1} {t}_{12} \\ {0}^{T} & 1\end{matrix} ]}^{- 1} [ \begin{matrix}{R}_{i} & α t {n}_{i} \\ 0 & 1\end{matrix} ] \\ [ \begin{matrix}{R}_{1} & {t}_{1} \\ 0 & 1\end{matrix} ]\end{matrix} \qquad ( 37 )$$

As a result, Rₚ is expressed by Equation (38), and tₚ is expressed by Equation (39).

Rₚ=R₁⁻¹R₁₂⁻¹R₁R₁ (38)

tₚ=R₁⁻¹R₁₂⁻¹R₁t₁−R₁⁻¹R₁₂⁻¹t₁₂−R₁⁻¹t₁−αR₁⁻¹R₁₂⁻¹tn₁ (39)

The calculating unit 121 then calculates coordinates (x(s)₁′, x(s)₂′, x(s)₃′) that are coordinates rotated from (x(s)₁, x(s)₂, x(s)₃) by Rₚ with Equation (40), using the third corresponding point group Q={(x(s)₁, x(s)₂, x(s)₃), (y(s)₁, y(s)₂, y(s)₃)}.

$$[ \begin{matrix}{x ( s )}_{1}^{′} \\ {x ( s )}_{2}^{′} \\ {x ( s )}_{3}^{′}\end{matrix} ] = {R}_{p} [ \begin{matrix}{x ( s )}_{1} \\ {x ( s )}_{2} \\ {x ( s )}_{3}\end{matrix} ] \qquad ( 40 )$$

The calculating unit 121 further calculates the difference tₚ′ between (x(s)₁′, x(s)₂′, x(s)₃′) and (y(s)₁, y(s)₂, y(s)₃) based on Equation (41).

$${t}_{P}^{′} = [ \begin{matrix}{y ( s )}_{1} \\ {y ( s )}_{2} \\ {y ( s )}_{3}\end{matrix} ] - [ \begin{matrix}{x ( s )}_{1}^{′} \\ {x ( s )}_{2}^{′} \\ {x ( s )}_{3}^{′}\end{matrix} ] \qquad ( 41 )$$

Among a plurality of tₚ calculated from tnᵢ with Equation (39), tₚ that is nearest to tₚ′ should be acquired. The scale of the vector remains undetermined because unknown α is included in tₚ in the Equation (39), but the orientation of the vector is determined. Hence, tnᵢ that is tₚ resulting in a vector nearest to tₚ′ and Rᵢ corresponding to that tnᵢ are determined as the desired R_{Vr} and tn_{Vr}.

Specifically, the calculating unit 121 will have Equation (42) by substituting tₚ=tₚ′ and rewriting Equation (39) for tnᵢ.

αtn₁=R₁₂R₁(R₁⁻¹R₁₂⁻¹R₁t₁−R₁⁻¹R₁₂⁻¹t₁₂−R₁⁻¹t₁−t′ₚ) (42)

Denoting the vector on the right side of Equation (42) by V, and denoting V normalized to a scale of one by Vn, the calculating unit 121 calculates the inner product of Vn and tnᵢ. The calculating unit 121 calculates the inner product of Vn and tnᵢ for each tnᵢ and determines one of tnᵢ resulting in an inner product with the absolute value nearest to one as tn_{Vr}, and establishes Rᵢ corresponding to the determined tnᵢ as R_{Vr}. The calculating unit 121 further calculates a value for unknown α from Equation (43), by substituting tnᵢ with tn_{Vr} in Equation (42).

α=V(n)/tn_{Vr}(n) (43)

Here, n is an element number in the vectors tn_{Vr} and V, and n takes a value from 1 to 3. Equation (43) may be calculated with n taking any one of 1 to 3, or Equation (43) may be calculated with n taking each one of 1 to 3, and the average of the results may be used as α. Alternatively, α may be calculated as the inner product of the vectors tn_{Vr} and V (α=dot(tn_{Vr}, V)).

The calculating unit 121 then calculates t_{Vr} from t_{Vr}=αtn_{Vr}. Given as a result is the virtual parameter C_{Vr}=[R_{Vr} t_{Vr}].

The calculating unit 121 then calculates the amount of rotational movement and the amount of translational movement of the subject (Step S611).

Specifically, the calculating unit 121 obtains a matrix [Rₚ tₚ] representing the amount of rotational movement and the amount of translational movement of the subject from Equations (38) and (39), and calculates a six-axis displacement parameter (ΔRₓ, ΔR_{y}, ΔR_{z}, Δtₓ, Δt_{y}, Δt_{z}) representing the rotational angle and the amount of translational movement of the subject in the X, Y, and Z axes from the obtained Rₚ and tₚ.

The rotational angle (ΔRₓ, ΔR_{y}, ΔR_{z}) can be calculated in the same manner as in the first embodiment. The amount of translational movement (Δtₓ, Δt_{y}, Δt_{z}) can be calculated with Equations (44) to (46).

Δtₓ=tₚ(1) (44)

Δt_{y}=tₚ(2) (45)

Δt_{z}=tₚ(3) (46)

Here, tₚ⁽¹⁾ to tₚ⁽³⁾ are the elements of tₚ.

The calculating unit 121 may select one of Rᵢ whose rotational angle about each of the X, Y, and Z axes is within an expected angle range, and tnᵢ corresponding to the selected Rᵢ as the desired R_{Vr} and tn_{Vr}.

When the appropriate five pairs are selected from the first group with RANSAC a plurality of number of times and the displacement calculating process is performed a plurality of number of times, the calculating unit 121 can calculate the amount of rotational movement and the amount of translational movement using one of Rᵢ and tnᵢ resulting in the inner product of Vn and tn_{Vr} nearest to one, such Rᵢ and to being obtained every time the displacement calculating process is performed, and use the calculated amounts of rotational movement and translational movement as the final displacement parameter (ΔRₓ, ΔR_{y}, ΔR_{z}, Δtₓ, Δt_{y}, Δt_{z}).

Referring back to FIG. 9, the control unit 123 controls to adjust the position of the subject to the position of the subject at the first point in time by rotating the bed on which the subject lies (not illustrated), using the difference (amount of rotational movement and the amount of translational movement) calculated by the calculating unit 121 (Step S411).

In the second embodiment, the bed can be rotated about and moved in parallel with the X axis, the Y axis, and the Z axis. The control unit 123 first rotates the bed about the Z axis by ΔR_{z}, then rotates the bed about the Y axis by ΔR_{y}, and finally rotates the bed about the X axis by ΔRₓ, using the amount of rotational movement calculated by the calculating unit 121. The control unit 123 then moves the bed by Δtₓ, Δt_{y}, and Δt_{z} in parallel in the X axis, the Y axis, and the Z axis, respectively. Parallel movements of the bed along these axes can be performed in any order.

The radiation unit 25 then irradiates a therapeutic beam to the affected area of the subject of which the position has been controlled in accordance with the irradiation information specified by the operator of the medical treatment apparatus 110 during the treatment planning (Step S413).

As described above, according to the second embodiment, because designations of corresponding point pairs performed on dissimilar images are minimized, influence of error in designations of the corresponding points in each pair can be reduced. Furthermore, the accuracy of the subject position control can be improved when the subject position control is performed for the amount of translational movement, as well as for the amount of rotational movement of the subject. Furthermore, according to the second embodiment, operators of the medical treatment apparatus 110 can easily designate pairs of corresponding points, so that burdens of the operators can be reduced.

Modification

Explained in a modification is an example in which the third corresponding point group Q has two or more pairs of corresponding points in the actual space at the first point in time and the second point in time in the example explained in the second embodiment. In this manner, the difference of the subject can be stably calculated, compared with when the third corresponding point group has only one pair of corresponding points.

In this example, the second group _P34 is represented as {(_p₁⁽³⁾, _p₁⁽⁴⁾), . . . , (_p_{L}⁽³⁾, _p_{L}⁽⁴⁾)} (L≧2), and the corresponding point group _P1234 is a group including the corresponding point pairs corresponding to those in the second group _P34 among those in the first group _P12, and including the point pairs in the second group _P34, that is, the corresponding point group _P1234 is represented as {(_pₛ⁽¹⁾, _pₛ⁽²⁾, _pₛ⁽³⁾, _pₛ⁽⁴⁾), . . . , (_p_{L}⁽¹⁾, _p_{L}⁽²⁾, _p_{L}⁽³⁾, _p_{L}⁽⁴⁾).

The calculating unit 121 obtains Qₛ{(x(m)₁, x(m)₂, x(m)₃), (y(m)₁, y(m)₂, y(m)₃)} for each case of m=1 to L, whereby acquiring the third corresponding point group Q={Q₁, . . . , Qₖ}, where k=L.

To calculate the virtual parameter C_{Vr}=[R_{Vr} t_{Vr}], the calculating unit 121 obtains tₚ′ given by Equation (41). To begin with, the calculating unit 121 calculates Equation (47) for every Q or for a plurality of Q{(x(k)₁, x(k)₂, x(k)₃), (y(k)₁, y(k)₂, y(k)₃)} in the third corresponding point group Q.

$${t}_{P}^{′} ( m ) = [ \begin{matrix}{y ( k )}_{1} \\ {y ( k )}_{2} \\ {y ( k )}_{3}\end{matrix} ] - [ \begin{matrix}{x ( k )}_{1}^{′} \\ {x ( k )}_{2}^{′} \\ {x ( k )}_{3}^{′}\end{matrix} ] \qquad ( 47 )$$

Here, (x(k)₁′, x(k)₂′, x(k)₃′) are coordinates in the actual space given by Equation (40). The calculating unit 121 then calculates tₚ′ using Equation (48)

$${t}_{P}^{′} = \frac{1}{m} {\sum }_{m} {t}_{p}^{′} ( m ) \qquad ( 48 )$$

After calculating tₚ′, the calculating unit 121 calculates the virtual parameter C_{Vr}=[R_{Vr} t_{Vr}] in the same manner as in the second embodiment.

In the manner described above, according to the first modification, the accuracy in estimating the difference of the subject is improved compared with when the third corresponding point group has only one corresponding point pair, so that the effectiveness of the medical treatment can be improved.

Hardware Configuration

FIG. 14 is a block diagram illustrating an example of a hardware configuration of the medical treatment apparatus according to the embodiments and the modification. As illustrated in FIG. 14, the medical treatment apparatus according to the embodiments and the modification includes a controller 902 such as a dedicated chip, a field programmable gate array (FPGA), or a central processing unit (CPU), a storage device 904 such as a read-only memory (ROM) and a random access memory (RAM), an external storage device 906 such a hard disk drive (HDD) or a solid state drive (SSD), a display device 908, input devices 910 such as a mouse and a keyboard, and a communication interface (I/F) 912, and can be implemented with a hardware configuration using a general computer.

The computer program executed on the medical treatment apparatus according to the embodiments and the modification is provided in a manner incorporated in the ROM or the like in advance. The computer program executed on the medical treatment apparatus according to the embodiments and the modification may also be provided in a manner recorded in a computer-readable recording medium such as a compact disc read-only memory (CD-ROM), a compact disc recordable (CD-R), a memory card, a digital versatile disc (DVD), or a flexible disk (FD) as an installable or executable file. The computer program executed on the medical treatment apparatus according to the embodiments and the modification may also be stored in a computer connected to a network such as the Internet, and may be made available for download over the network.

The computer program executed on the medical treatment apparatus according to the embodiments and the modification has a modular structure that allows a computer to implement each of the units described above. In the actual hardware, for example, the controller 902 reads the computer program from the external storage device 906 onto the storage device 904, and executes the computer program to implement each of the units described above on the computer.

In the manner described above, according to the embodiments and the modification, the accuracy of the subject position control can be improved.

The steps in the flowchart according to the embodiment may be executed in a different order, or some of the steps may be executed simultaneously as long as such a modification is not against the nature of the process. The steps may also be executed in a different order every time the process is executed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical treatment apparatus comprising:
a first acquirer that acquires a first group including five or more pairs of corresponding points respectively on a first perspective image of a subject viewed in a first direction at a first timing and a second perspective image of the subject viewed in a second direction at a second timing being different from the first timing;
a second acquirer that acquires a first parameter and a second parameter, the first parameter including position and orientation information of an imaging device that captures the first perspective image and including conversion information related to a coordinate system of the first perspective image, and the second parameter including position and orientation information of an imaging device that captures the second perspective image and including conversion information related to a coordinate system of the second perspective image;
a calculator to calculate difference in position of the subject between the first timing and second timing using the first group, the first parameter, and the second parameter; and
a controller that controls a position of the subject using the difference.

2. The apparatus according to claim 1, wherein
the first acquirer further acquires a second group that includes a pair of corresponding points respectively on a third perspective image of the subject viewed in a third direction at the first timing and a fourth perspective image of the subject viewed in a third direction at the first timing, and at least one of the pair included in the second group is corresponding to the pair included in the first group,
the second acquirer further acquires a third parameter and a fourth parameter, the third parameter including position and orientation information of an imaging device that captures the third perspective image and including conversion information related to a coordinate system of the third perspective image, and the fourth parameter including position and orientation information of an imaging device that captures the fourth perspective image and including conversion information related to a coordinate system of fourth perspective image, and
the calculator calculates the difference additionally using the second group, the third parameter, and the fourth parameter.

3. The apparatus according to claim 1, wherein the difference is represented as an amount of rotational movement of the subject from the first timing to the second timing.

4. The apparatus according to claim 3, wherein
the difference further includes a direction of translational movement of the subject from the first timing to the second timing,
the direction of translational movement is a direction of translational movement of the subject at the second timing with respect to the position of the subject at the first timing, and
the controller controls the position of the subject using the amount of rotational movement and the direction of translational movement.

5. The apparatus according to claim 1, further comprising a display to display the first perspective image and the second perspective image, wherein
the first group includes five or more pairs of corresponding points entered by an operator on the first perspective image and the second perspective image displayed on the display.

6. The apparatus according to claim 2, wherein the difference is represented as an amount of rotational movement and an amount of translational movement of the subject from the first timing to the second timing.

7. The apparatus according to claim 6, wherein
the second timing is time subsequent to the first timing,
the amount of rotational movement is an amount of rotational movement of the subject at the second timing with respect to the position of the subject at the first timing,
the amount of translational movement is an amount of translational movement of the subject at the second timing with respect to the position of the subject at the first timing, and
the controller controls the position of the subject using the amount of rotational movement and the amount of translational movement.

8. The apparatus according to claim 2, further comprising a display to display the first perspective image, the second perspective image, the third perspective image, and the fourth perspective image, wherein
the first group includes five or more pairs of corresponding points entered by an operator on the first perspective image and the perspective second image displayed on the display, and
the second group includes one or more pairs of corresponding points entered by an operator on the third perspective image and the fourth perspective image displayed on the display.

9. The apparatus according to claim 6, wherein
the calculator calculates a third group that is a group of one or more pairs of corresponding points from the first timing to the second timing in an actual space, using a pair included in the first group that corresponds to a pair included in the second group, the second group, and the first to fourth parameters,
the calculator calculates a virtual parameter related to position and orientation of the imaging device that captures the first perspective image and another imaging device assumed to have captured the second perspective image of the subject at same position as that at the first timing, using the first group and the third group, and
the calculator calculates the amount of rotational movement and the amount of translational movement using the virtual parameter.

10. The apparatus according to claim 1, further comprising a radiator that irradiates a therapeutic beam to the subject of which the position has been controlled.

11. The apparatus according to claim 2, wherein
the imaging device that captures the first perspective image is the same imaging device that captures the third perspective image,
the imaging device that captures the second perspective image is the same imaging device that captures the fourth perspective image, and
the imaging device that captures the first and third perspective images is different from the imaging device that captures the second and fourth perspective images.

12. The apparatus according to claim 2, wherein
the first direction is approximately the same as the second direction, and
the third direction is approximately the same as the fourth direction.

13. a control device comprising:
a processor; and
a memory that stores processor-executable instructions that, when executed by the processor, cause the processor to:
acquiring a first group including five or more pairs of corresponding points respectively on a first perspective image of a subject viewed in a first direction at a first timing and a second perspective image of the subject viewed in a second direction at a second timing being different from the first timing;
acquiring a first parameter and a second parameter, the first parameter including position and orientation information of an imaging device that captures the first perspective image and including conversion information related to a coordinate system of the first perspective image, and the second parameter including position and orientation information of an imaging device that captures the second perspective image and including conversion information related to a coordinate system of the second perspective image;
calculating difference in position of the subject between the first timing and second timing using the first group, the first parameter, and the second parameter; and
controlling a position of the subject using the difference.

14. A control method comprising:
acquiring a first group including five or more pairs of corresponding points respectively on a first perspective image of a subject viewed in a first direction at a first timing and a second perspective image of the subject viewed in a second direction at a second timing being different from the first timing;
acquiring a first parameter and a second parameter, the first parameter including position and orientation information of an imaging device that captures the first perspective image and including conversion information related to a coordinate system of the first perspective image, and the second parameter including position and orientation information of an imaging device that captures the second perspective image and including conversion information related to a coordinate system of the second perspective image;
calculating difference in position of the subject between the first timing and second timing using the first group, the first parameter, and the second parameter; and
controlling a position of the subject using the difference.
